(19) United States
(12) Patent Application Publication
(10) Pub. No.: US 2026/0295237 A1
(43) Pub. Date: Oct. 1, 2026

(54) SYNCHRONIZATION OF A MEASUREMENT TO AN EXTERNAL SIGNAL

(71) Applicants: Abiomed, Inc., Danvers, MA (US); Abiomed Europe GmbH, Aachen (DE)

(72) Inventors: Claude Belleville, Danvers, MA (US); Andrew Gentile, Danvers, MA (US); Johannes Visser, Aachen (DE)

(73) Assignees: Abiomed, Inc., Danvers, MA (US); Abiomed Europe GmbH, Aachen (DE)

(21) Appl. No.: 19/576,146

(22) Filed: Mar. 24, 2026

Related U.S. Application Data
(60) Provisional application No. 63/779,747, filed on Mar. 28, 2025.

(51) Int. Cl. A61M 60/554; A61M 60/165; A61M 60/216; A61M 60/816

(52) U.S. Cl. CPC A61M 60/554; A61M 60/165; A61M 60/216; A61M 60/816; A61M 2205/103; A61M 2205/3303; A61M 2205/3306; A61M 2205/3327; A61M 2205/3344; A61M 2205/50; A61M 2210/125; A61M 2230/30

(57) ABSTRACT

Methods and apparatus for performing a pressure measurement associated with a heart pump are described. The method includes controlling a light source of an optical pressure sensor to generate one or more light pulses in synchronization with a signal received from pump drive circuitry associated with the heart pump, capturing an image based on an optical signal generated based, at least in part, on the one or more light pulses, and determining a pressure based, at least in part, on the image.

### RELATED APPLICATIONS

This application claims benefit under 35 U.S.C. § 119(e) of U.S. Provisional application serial number 63/779,747, filed Mar. 28, 2025, the disclosure of which is incorporated by reference herein in its entirety.

### BACKGROUND

Fluid pumps, such as blood pumps, are used in the medical field in a wide range of applications and purposes. An intravascular blood pump is a pump that can be advanced through a patient’s vasculature, i.e., veins and/or arteries, to a position in the patient’s heart or elsewhere within the patient’s circulatory system. For example, an intravascular blood pump may be inserted via a catheter and positioned to span a heart valve. The intravascular blood pump is typically disposed at the end of the catheter. Once in position, the pump may be used to assist the heart and pump blood through the circulatory system and, therefore, temporarily reduce workload on the patient’s heart, such as to enable the heart to recover after a heart attack. An exemplary intravascular blood pump is available from ABIOMED, Inc., Danvers, MA under the tradename Impella^{®} heart pump.

Such pumps can be positioned, for example, in a cardiac chamber, such as the left ventricle, to assist the heart. In this case, the blood pump may be inserted via a femoral artery by means of a hollow catheter and introduced up to and into the left ventricle of a patient’s heart. From this position, the blood pump inlet draws in blood and the blood pump outlet expels the blood into the aorta. In this manner, the heart’s function may be replaced or at least assisted by operation of the pump.

An intravascular blood pump is typically connected to a respective external heart pump controller that controls the heart pump, such as motor speed, and collects and displays operational data about the blood pump, such as heart signal level, battery temperature, blood flow rate and plumbing integrity. An exemplary heart pump controller is available from ABIOMED, Inc. under the trade name Automated Impella Controller^{®}. The controller may raise alarms when operational data values fall beyond predetermined values or ranges, for example if a leak, suction, and/or pump malfunction is detected. The controller may include a video display screen upon which is displayed a graphical user interface configured to display the operational data and/or alarms.

### SUMMARY

Described herein are systems and methods for synchronizing a measurement using a sensor (e.g., an optical pressure sensor) of a medical device (e.g., a heart pump) to a signal associated with operation of the medical device. The synchronization of the measurement with the signal may enable a measurement that is less influenced by the operating conditions of the medical device. For example, synchronizing a timing of a light pulse provided to an optical pressure sensor of a heart pump with a motor drive signal of the heart pump may enable the measurement of a pressure value with high accuracy.

In some embodiments, a method of performing a pressure measurement associated with a heart pump is provided. The method includes controlling a light source of an optical pressure sensor to generate one or more light pulses in synchronization with a signal received from pump drive circuitry associated with the heart pump, capturing an image based on an optical signal generated based, at least in part, on the one or more light pulses, and determining a pressure based, at least in part, on the image.

In one aspect, the signal reflects a blade position of an impeller driven by the pump drive circuitry. In another aspect, the signal comprises a signal received from a transistor gate of the pump drive circuitry. In another aspect, the one or more light pulses comprises a plurality of light pulses that collectively produce stroboscopic flashing by the light source. In another aspect, the light source is a light emitting diode (LED) source. In another aspect, the method further includes selecting one or more characteristics of the one or more light pulses based, at least in part, on a speed of a motor associated with the pump drive circuitry. In another aspect, the one or more characteristics include one or more of a pulse width, a pulse delay, or a pulse amplitude.

In some embodiments, a mechanical circulatory support system is provided. The mechanical circulatory support device includes a heart pump including an impeller and at least one optical pressure sensor, pump drive circuitry configured to drive rotation of the impeller, and a controller. The controller is configured to generate a synchronization signal based, at least in part, on a signal received from the pump drive circuitry, control operation of one or more light sources to output one or more light pulses to the at least one optical pressure sensor, capture an image based on an optical signal generated by the at least one optical pressure sensor in response to the at least one optical pressure sensor receiving the one or more light pulses, and determine a pressure based, at least in part, on the image.

In one aspect, the signal reflects a blade position of the impeller. In another aspect, the signal comprises a signal received from a transistor gate of the pump drive circuitry. In another aspect, the one or more light pulses comprises a plurality of light pulses that collectively produce stroboscopic flashing by the one or more light sources. In another aspect, the one or more light sources include a light emitting diode (LED) source. In another aspect, the controller is further configured to select one or more characteristics of the one or more light pulses based, at least in part, on a speed of a motor associated with the pump drive circuitry. In another aspect, the one or more characteristics include one or more of a pulse width, a pulse delay, or a pulse amplitude.

In some embodiments, a controller for a mechanical circulatory support system is provided. The controller includes at least one hardware processor programmed with instructions that, when executed, perform a process. The process includes generating a synchronization signal based, at least in part, on a pump drive signal, controlling operation of one or more light sources to output one or more light pulses to at least one optical sensor, capturing an image based on an optical signal generated by the at least one optical sensor in response to the at least one optical sensor receiving the one or more light pulses, and determining a pressure based, at least in part, on the image.

In one aspect, the pump drive signal reflects a blade position of an impeller of the mechanical circulatory support system. In another aspect, the pump drive signal comprises a signal received from a transistor gate of motor drive circuitry of the mechanical circulatory support system. In another aspect, the one or more light pulses comprises a plurality of light pulses that collectively produce stroboscopic flashing by the one or more light sources. In another aspect, the one or more light sources include a light emitting diode (LED) source. In another aspect, the process further includes selecting one or more characteristics of the one or more light pulses based, at least in part, on a speed of a motor associated with the mechanical circulatory support system, wherein the one or more characteristics include one or more of a pulse width, a pulse delay, or a pulse amplitude.

### BRIEF DESCRIPTION OF DRAWINGS

FIG. 1A shows an illustrative circulatory support device that may be used in accordance with some embodiments.

FIG. 1B illustrates the circulatory support device of FIG. 1A positioned within a heart of a patient.

FIG. 1C illustrates a ventricular support system including the circulatory support device of FIG. 1A.

FIG. 2 illustrates a circulatory support device including a multi-channel optical pressure sensor in accordance with some embodiments.

FIG. 3 schematically illustrates distortion of a pressure measurement signal based on blade position of a rotating impeller of a heart pump, in accordance with some embodiments.

FIG. 4A is a timing diagram of an image capture sequence for an optical pressure sensor of a heart pump.

FIG. 4B is a timing diagram of an image capture sequence for an optical pressure sensor of a heart pump in which a light pulse generated during the image capture sequence is synchronized with a motor drive signal of the heart pump, in accordance with some embodiments.

FIG. 5A shows an example of an LED pulse having first characteristics being synchronized to a motor drive signal, in accordance with some embodiments.

FIG. 5B shows an example of an LED pulse having second characteristics being synchronized to a motor drive signal, in accordance with some embodiments.

FIG. 6 is a control system architecture for a pressure sensing system of a heart pump, in accordance with some embodiments.

### DETAILED DESCRIPTION

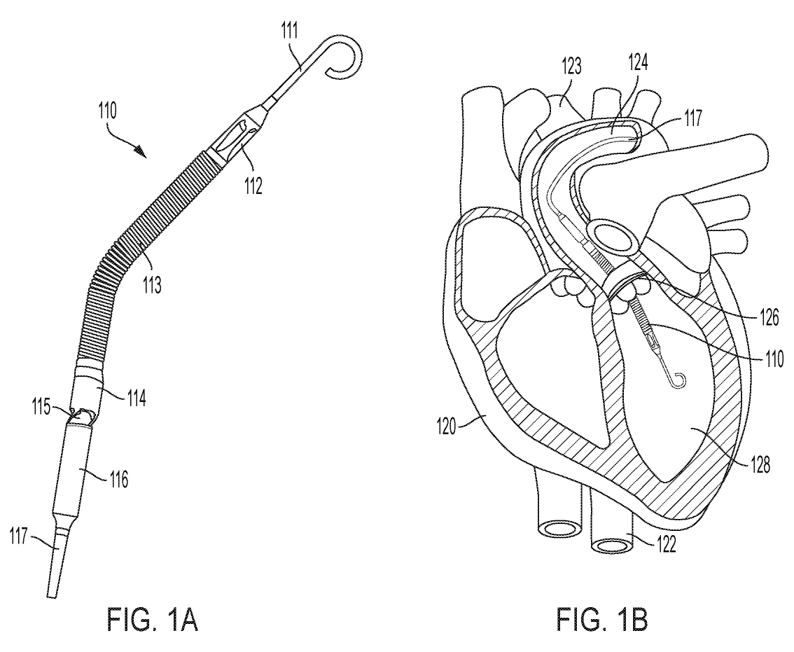

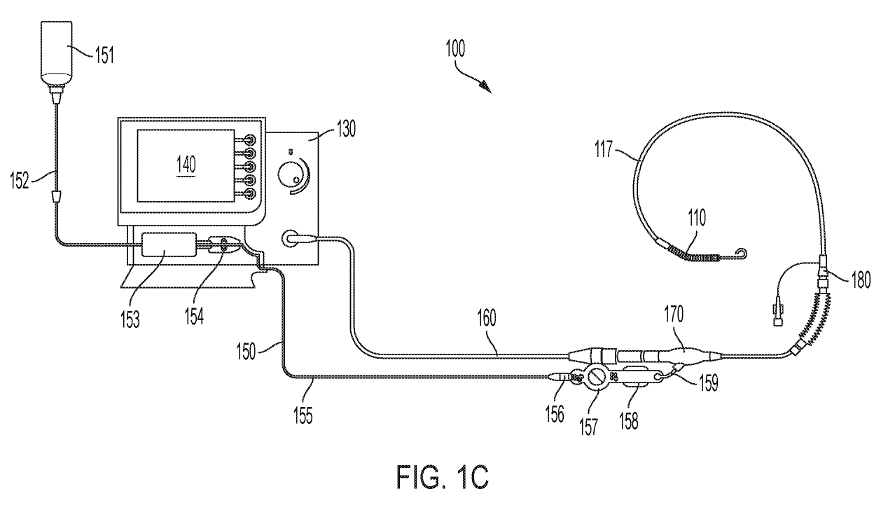

A circulatory support device (also referred to herein as a “heart pump” or simply a “pump”) may include a percutaneous, catheter-based device that provides hemodynamic support to the heart of a patient. As will be appreciated, for a heart pump to function properly, it should be positioned correctly in the heart of a patient. For a heart pump inserted in the left side of the heart, proper positioning may include an inlet portion of the pump located in the left ventricle and an outlet portion of the pump located in the aorta, thereby spanning the aortic valve of the patient’s heart. As shown in FIG. 1A, a heart pump 110 may include a pigtail 111, an inlet area 112, a cannula 113, a pressure sensor 114, an outlet area 115, a motor housing 116, and/or a catheter tube 117. Pigtail 111 may assist with stabilizing heart pump 110 in the heart of a patient. It should be appreciated that some embodiments of heart pump 110 may not include pigtail 111 and heart pump 110 may be stabilized in other ways or not at all. During operation, blood may be drawn into one or more openings of inlet area 112, channeled through cannula 113, and expelled through one or more openings of outlet area 115 by a motor (not shown) disposed in motor housing 116. The motor may drive rotation of an impeller that includes blades that rotate to pump the blood from the inlet area 112 to the outlet area 115. In some implementations, pressure sensor 114 may include a flexible membrane that is integrated into cannula 113. One side of pressure sensor 114 may be exposed to the blood pressure on the outside of cannula 113, and the other side may be exposed to the pressure of the blood inside of cannula 113. In some such implementations, pressure sensor 114 may generate an electrical signal proportional to the difference between the pressure outside cannula 113 and the pressure inside cannula 113. In some implementations, pressure sensor 114 may include an optical pressure sensor. Catheter tube 117 may provide one or more fluidic and/or electrical connections between heart pump 110 and one or more other devices of a ventricular support system, an example of which is shown in FIG. 1C.

As shown in FIG. 1B, heart pump 110 may be positioned in a patient's heart 120. For example, heart pump 110 may be inserted percutaneously via the femoral artery 122 into the ascending aorta 124, across the aortic valve 126, and into the left ventricle 128. In other implementations, a heart pump may, for example, be inserted percutaneously via the axillary artery 123 into the ascending aorta 124, across the aortic valve 126, and into the left ventricle 128. In other implementations, a heart pump may, for example, be inserted directly into the ascending aorta 124, across the aortic valve 126, and into the left ventricle 128. During operation, heart pump 110 may entrain blood from the left ventricle 128 and expel blood into the ascending aorta 124. As a result, heart pump 110 may perform some of the work normally done by the patient's heart 120. The hemodynamic effects of heart pumps may include an increase in cardiac output, improvement in coronary blood flow resulting in a decrease in left ventricle end-diastolic pressure, pulmonary capillary wedge pressure, myocardial workload, and oxygen consumption.

As shown in FIG. 1C, heart pump 110 may form part of a ventricular support system 100. Ventricular support system 100 also may include a controller 130 (e.g., an Automated Impella Controller^{®}, referred to herein as “AIC,” from ABIOMED, Inc., Danvers, Mass.), a display 140, a purge subsystem 150, a connector cable 160, a plug 170, and a repositioning unit 180. As shown, controller 130 may include display 140. Controller 130 monitors and controls operation of heart pump 110. During operation, purge subsystem 150 may be configured to deliver a purge fluid to heart pump 110 through catheter tube 117 to prevent blood from entering the motor (not shown) within motor housing 116. In some implementations, the purge fluid is a dextrose solution (e.g., 5% dextrose in water with 25 or 50 IU/mL of heparin). Connector cable 160 may provide an electrical connection between heart pump 110 and controller 130. Plug 170 may connect catheter tube 117, purge subsystem 150, and connector cable 160. In some implementations, plug 170 includes a storage device (e.g., a memory) configured to store, for example, operating parameters to facilitate transfer of the patient to another controller if needed. Repositioning unit 180 may be used to reposition heart pump 110 in the patient’s heart.

As shown, in some embodiments, the ventricular support system may include a purge subsystem 150 having a container 151, a supply line 152, a purge cassette 153, a purge disc 154, purge tubing 155, a check valve 156, a pressure reservoir 157, an infusion filter 158, and a sidearm 159. Container 151 may, for example, be a bag or a bottle. As will be appreciated, in other embodiments the ventricular support system may not include a purge subsystem. In some embodiments, a purge fluid may be stored in container 151. Supply line 152 may provide a fluidic connection between container 151 and purge cassette 153. Purge cassette 153 may control how the purge fluid in container 151 is delivered to heart pump 110. For example, purge cassette 153 may include one or more valves for controlling a pressure and/or flow rate of the purge fluid. Purge disc 154 may include one or more pressure and/or flow sensors for measuring a pressure and/or flow rate of the purge fluid. As shown, controller 130 may include purge cassette 153 and purge disc 154. Purge tubing 155 may provide a fluidic connection between purge disc 154 and check valve 156. Pressure reservoir 157 provides additional filling volume during a purge fluid change. In some implementations, pressure reservoir 157 includes a flexible rubber diaphragm that provides the additional filling volume by means of an expansion chamber. Infusion filter 158 helps prevent bacterial contamination and air from entering catheter tube 117. Sidearm 159 provides a fluidic connection between infusion filter 158 and plug 170.

Although shown as having separate purge tubing and connector cable, it will be appreciated that in some embodiments, the ventricular support system may include a single connector with both fluidic and electric lines connectable to the AIC.

During operation, controller 130 may be configured to receive measurements from pressure sensor 114 and purge disc 154 and to control operation of the motor (not shown) within motor housing 116 and purge cassette 153. As noted above, controller 130 may be configured to control and measure a pressure and/or flow rate of a purge fluid via purge cassette 153 and purge disc 154. During operation, after exiting purge subsystem 150 through sidearm 159, the purge fluid may be channeled through purge lumens (not shown) within catheter tube 117 and plug 170. Sensor cables (not shown) within catheter tube 117, connector cable 160, and plug 170 may provide an electrical connection between pressure sensor 114 and controller 130. Motor cables (not shown) within catheter tube 117, connector cable 160, and plug 170 may provide an electrical connection between the motor within motor housing 116 and controller 130. During operation, controller 130 may be configured to receive measurements from pressure sensor 114 through the sensor cables (e.g., optical fibers) and to control the electrical power delivered to the motor within motor housing 116 through the motor cables. By controlling the power delivered to the motor within motor housing 116, controller 130 is operable to control the speed of the motor within motor housing 116.

Various modifications can be made to ventricular support system 100 and one or more of its components. For instance, one or more additional sensors may be added to ventricular support system 100. In another example, a signal generator may be added to ventricular support system 100 to generate a signal indicative of the rotational speed of the motor within motor housing 116. As another example, one or more components of ventricular support system 100 may be separated. For instance, display 140 may be incorporated into another device in communication with controller 130 (e.g., wirelessly or through one or more electrical cables).

As described herein, a heart pump (e.g., heart pump 110) may include a pressure sensor 114 (e.g., an optical pressure sensor) configured to detect a pressure within the aorta of a patient’s heart when the heart pump is properly positioned. The pressure signal sensed by pressure sensor 114 may be used, at least in part, to determine correct positioning of the heart pump within the patient’s heart and/or to determine a blood flow rate through the heart pump when in operation. For instance, the pressure signal may be used in combination with a motor current signal received from a motor current sensor (not shown) and a set of stored values to determine a flow rate through the heart pump. The differential pressure across the aortic valve may also indirectly be determined based on the pressure signal measuring the pressure in the aorta and the set of stored values.

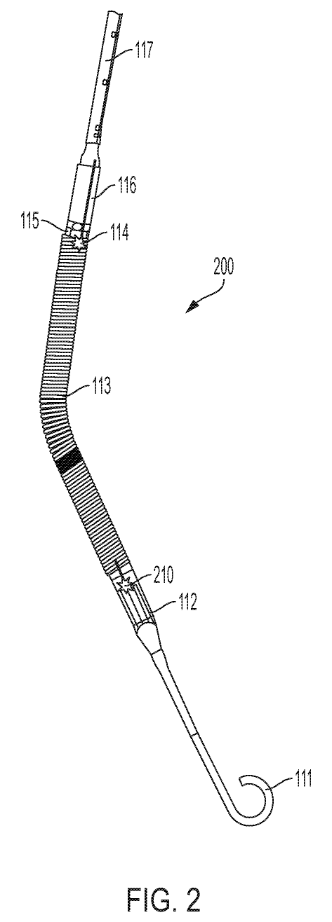

The inventors have recognized and appreciated that it may be useful to incorporate one or more additional pressure sensors in heart pump 110, for example, to directly sense pressure in both the aorta and the left ventricle rather than having to infer the pressure in the left ventricle based on the pressure sensor signal sensed in the aorta, as discussed above. Use of multiple pressure sensors is also referred to herein as implementing a multi-channel pressure sensor. FIG. 2 illustrates an embodiment of heart pump 200 in which a second pressure sensor 210 is arranged near inlet area 112. When properly positioned within the left side of the heart of a patient, the second pressure sensor 210 may be configured to measure a pressure sensor signal used to determine a left ventricular blood pressure. In such implementations, additional sensor cables (e.g., optical fibers) may be disposed within catheter tube 117 to provide a connection between the second pressure sensor 210 and the controller (e.g., controller 130).

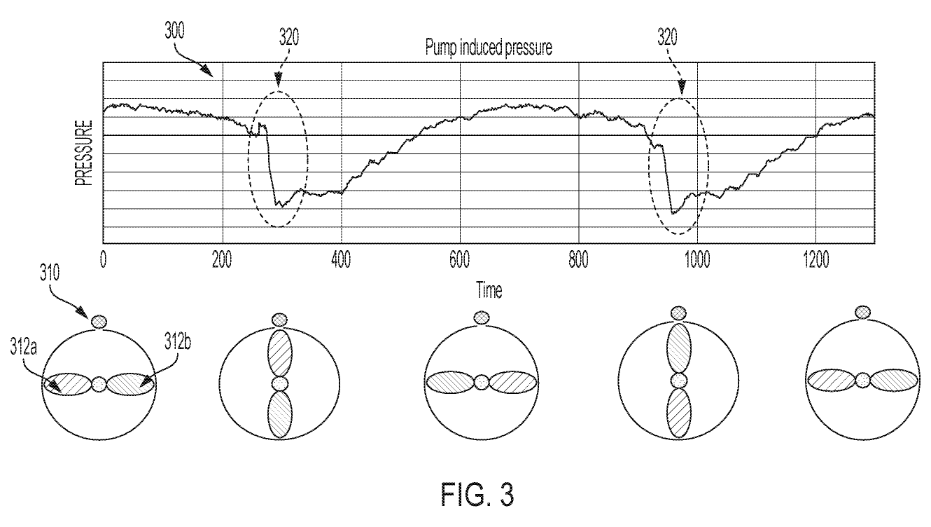

The inventors have recognized that measurements sensed by some sensors associated with (e.g., included on) a medical device (e.g., pressure sensor 114 and/or second pressure sensor 210 included on heart pump 200) may be influenced by the operation of the medical device itself. In the example of a heart pump that includes one or more optical pressure sensors, the pressure measurement(s) sensed by the sensors may be impacted by the blade position of the rotating impeller when the measurement is acquired. FIG. 3 schematically illustrates how a pressure signal 300 sensed by an optical pressure sensor may be influenced by the blade position of the impeller of the heart pump during a rotation cycle of the impeller. For example, FIG. 3 shows that when the blades 312a, 312b of the impeller are not located near a sensor (e.g., not near a casing opening where pressure sensor 310 is located), the pressure signal 300 is relatively stable. However, as the impeller rotates and one of blades 312a, 312b passes near the sensor, the pressure signal may be disturbed. For example, in the illustrative embodiments shown in FIG. 3, a large negative spike in the pressure signal 320 is observed. When the pressure signal 300 is integrated over multiple impeller rotation cycles, the measured pressure signal over the integration period may be less than the actual value of the pressure in the heart due to the disturbances caused by the blade passages near the sensor 310.

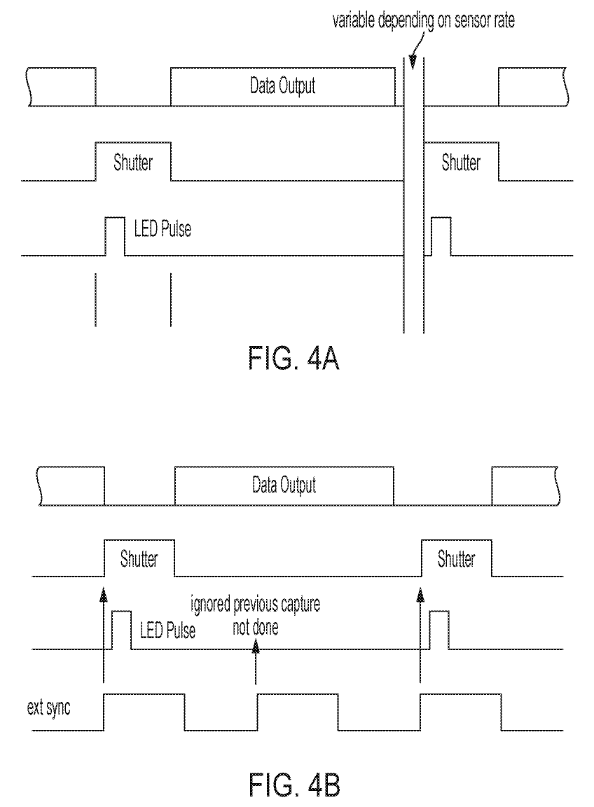

Some embodiments of the present disclosure relate to systems and methods for synchronizing the timing of a measurement period of a sensor (e.g., an optical pressure sensor) with a signal associated with the operation of the medical device (e.g., a heart pump) on which the sensor is located. Referring to the example shown in FIG. 3, the pressure measurement may be determined by sensing the pressure during times in which the pressure signal is relatively stable (e.g., when one of the blades is not near the sensor) and refraining from sensing the pressure during times in which the pressure signal is disturbed due to the blades passing the sensor. In the example of an optical pressure sensor, the pressure measurement may be obtained by transmitting a light pulse from a light source (e.g., a light emitting diode (LED)) and capturing an image based on light reflected by the optical pressure sensor. FIG. 4A shows a timing diagram of an image capture sequence for an optical pressure sensor. In the example of FIG. 4A, the start of a fixed shutter period (e.g., 1 ms, 2 ms, 3 ms, 5 ms, etc.) is timed with a fixed pace (e.g., 5 ms, 10 ms, 20 ms, 40 ms). The shutter period may represent the time during which the shutter of a camera including an image sensor is open, which may also be referred to as an “integration period.” An LED pulse is flashed within the shutter period and the width of the LED pulse may be selected to control the brightness of the image captured during a period following the shutter period. As illustrated in FIG. 4A, the timing between the completion of image capture period and the beginning of the next shutter period may be variable (e.g., random) and may depend, for example, on characteristics of the image sensor hardware. In the example of FIG. 4A because the start of the capture/measurement cycle is not synchronized to any particular event, the LED pulse flash may happen in some measurements during times in which a blade of the impeller is near the sensor, thereby distorting the measured pressure signal as described herein.

The inventors have recognized and appreciated that synchronizing the occurrence of the light pulse (e.g., LED pulse flash) with a signal that reflects the blade position of the impeller may enable for capture/measurement of a pressure signal with reduced distortion. FIG. 4B shows a timing diagram of an image capture sequence for an optical pressure sensor in accordance with some embodiments. In the example of FIG. 4B, the start of a shutter period is synchronized based on an external signal (e.g., a synchronization signal) that reflects the motor operation (and thus the blade position) of the heart pump. Similar to the timing diagram shown in FIG. 4A, an LED pulse is flashed at a fixed delay relative to the start of the shutter period. In this way, the LED pulse is also synchronized to the external signal. Rather than initiating a next capture/measurement period at a variable time (e.g., based on the sensor hardware characteristics), as shown in the example of FIG. 4A, the timing diagram of FIG. 4B shows that the next capture/measurement period is synchronized to the pulses in the external signal to ensure that the next capture/measurement is aligned with a motor operation timing. The synchronization of the capture/measurement period with the external signal ensure that the LED pulse flash occurs during a time in which a blade of the impeller is not passing by the pressure sensor. As shown in FIG. 4B, pulses in the external signal that occur during an image capture may be ignored and the next capture/measurement cycle may begin based on a synchronization with the next pulse in the external signal that follows the completion of the image capture. In the example shown in FIG. 4B, the start of a capture/measurement cycle is synchronized to a rising edge of the external signal. It should be appreciated however, that the start of a capture/measurement period may be synchronized with the external signal in any other suitable way (e.g., synchronized to the falling edge of a synchronization pulse).

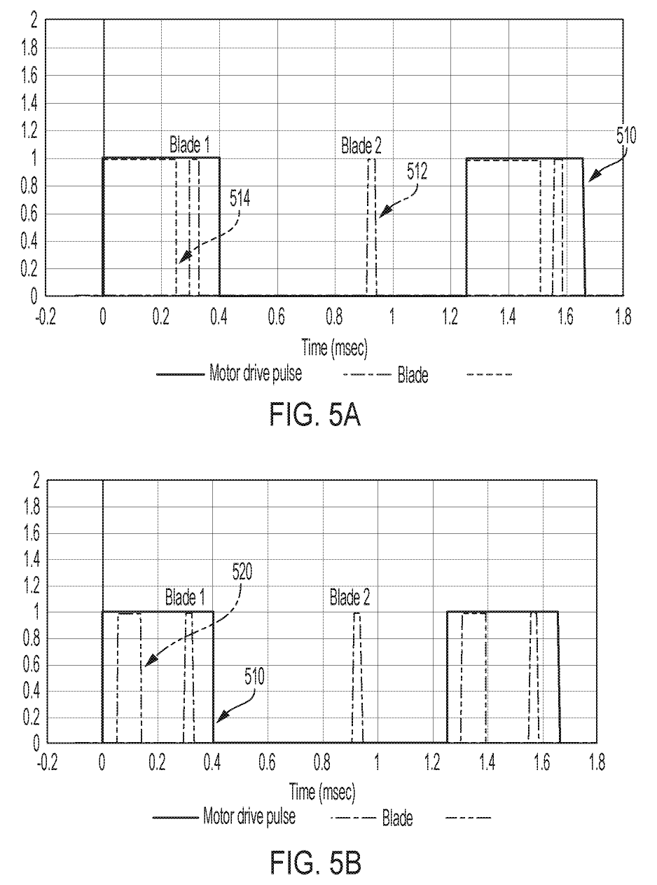

In some embodiments, the external signal used to synchronize the timing of the start of an LED pulse for a pressure sensor may be based on one or more metrics indicative of a medical device. For example, in embodiments disclosed herein with a heart pump having a sensor, a motor drive signal sensed from motor driving circuitry of the heart pump could be used to determine the appropriate synchronization and start time for the LED pulse. FIG. 5A illustrates a timing diagram showing the relative timing between a motor drive pulse signal 510 received from motor drive circuitry of a heart pump, a blade proximity signal 512 illustrating a relative location of the blade of the impeller to the optical pressure sensor during a rotation cycle, and an LED pulse 514. In some embodiments, one or more characteristics of the LED pulse 514 may be adjustable or tunable. For example, the timing diagram in FIG. 5A shows an LED pulse 514 with a first pulse width and a start time that aligns with the rising edge of a motor drive pulse in the motor drive pulse signal 510 (also referred to as having a delay of 0 ms). By contrast, the timing diagram in FIG. 5B shows an LED pulse 520 with a second pulse width narrower than the first pulse width of LED pulse 514 and a start time that is slightly delayed from the rising edge of a motor drive pulse in the motor drive pulse signal 510. It should be appreciated that the LED pulse characteristics shown in FIGS. 5A and 5B are merely examples, and any suitable LED pulse characteristics may be used.

As can be appreciated, although the wider LED pulse 514 of FIG. 5A may result in increased illumination, and thus a brighter captured image compared with the LED pulse 520 of FIG. 5B, the wider LED pulse 514 also provides illumination closer in time to when the blade position is passing the optical pressure sensor, which may distort the pressure measurement, as described herein. Accordingly, in some embodiments, use of a narrower (and possibly high intensity) LED pulse, such as that shown in FIG. 5B may result in a pressure measurement signal with less distortion due to an undesirable blade position during LED pulse flashing. It should be appreciated that an LED pulse with any suitable characteristics (e.g., delay, width, intensity) may be used. In some embodiments, the characteristics of the LED pulse may be selected based, at least in part, on a speed of the motor driving pump operation.

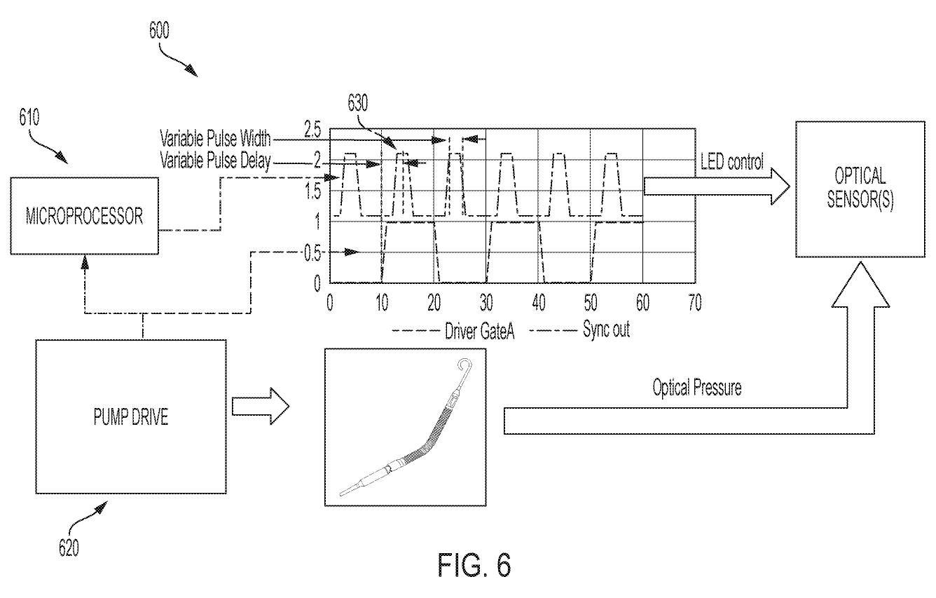

In some embodiments, examples of which are described above, individual LED pulses during a capture/measurement cycle of an optical pressure sensor may be synchronized to an external signal (e.g., a signal generated from motor drive circuitry). In some embodiments, the LED pulse may be controlled to strobe at a timing based on the motor drive signal. FIG. 6 illustrates a control system architecture 600 in which microprocessor 610 receives a signal from pump drive circuitry 620. For instance, the signal received from pump drive circuitry 620 may be a signal from a transistor gate of pump drive circuitry 620 that reflects the impeller blade position during a rotation of the impeller. Based on the received signal, microprocessor 610 may generate a series of LED control pulses 630 synchronized with the signal received from the pump drive circuitry 620. As shown in FIG. 6, microprocessor 610 may be configured to generate LED control pulses 630 having various pulse widths and/or pulse delays relative to the motor drive signal received from the pump drive circuitry 620. The LED control pulses may be used to control the operation of one or more LED sources to generate one or more LED pulses as part of an image capture sequence for an optical pressure sensor measurement as described herein.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A method of performing a pressure measurement associated with a heart pump, the method comprising:
controlling a light source of an optical pressure sensor to generate one or more light pulses in synchronization with a signal received from pump drive circuitry associated with the heart pump;
capturing an image based on an optical signal generated based, at least in part, on the one or more light pulses; and
determining a pressure based, at least in part, on the image.

2. The method of claim 1, wherein the signal reflects a blade position of an impeller driven by the pump drive circuitry.

3. The method of claim 1, wherein the signal comprises a signal received from a transistor gate of the pump drive circuitry.

4. The method of claim 1, wherein the one or more light pulses comprises a plurality of light pulses that collectively produce stroboscopic flashing by the light source.

5. The method of claim 1, wherein the light source is a light emitting diode (LED) source.

6. The method of claim 1, further comprising:
selecting one or more characteristics of the one or more light pulses based, at least in part, on a speed of a motor associated with the pump drive circuitry.

7. The method of claim 6, wherein the one or more characteristics include one or more of a pulse width, a pulse delay, or a pulse amplitude.

8. A mechanical circulatory support system comprising:
a heart pump including an impeller and at least one optical pressure sensor;
pump drive circuitry configured to drive rotation of the impeller; and
a controller configured to:
generate a synchronization signal based, at least in part, on a signal received from the pump drive circuitry;
control operation of one or more light sources to output one or more light pulses to the at least one optical pressure sensor;
capture an image based on an optical signal generated by the at least one optical pressure sensor in response to the at least one optical pressure sensor receiving the one or more light pulses; and
determine a pressure based, at least in part, on the image.

9. The mechanical circulatory support system of claim 8, wherein the signal reflects a blade position of the impeller.

10. The mechanical circulatory support system of claim 8, wherein the signal comprises a signal received from a transistor gate of the pump drive circuitry.

11. The mechanical circulatory support system of claim 8, wherein the one or more light pulses comprises a plurality of light pulses that collectively produce stroboscopic flashing by the one or more light sources.

12. The mechanical circulatory support system of claim 8, wherein the one or more light sources include a light emitting diode (LED) source.

13. The mechanical circulatory support system of claim 8, wherein the controller is further configured to:
select one or more characteristics of the one or more light pulses based, at least in part, on a speed of a motor associated with the pump drive circuitry.

14. The mechanical circulatory support system of claim 13, wherein the one or more characteristics include one or more of a pulse width, a pulse delay, or a pulse amplitude.

15. A controller for a mechanical circulatory support system, the controller comprising:
at least one hardware processor programmed with instructions that, when executed, perform a process comprising:
generating a synchronization signal based, at least in part, on a pump drive signal;
controlling operation of one or more light sources to output one or more light pulses to at least one optical sensor;
capturing an image based on an optical signal generated by the at least one optical sensor in response to the at least one optical sensor receiving the one or more light pulses; and
determining a pressure based, at least in part, on the image.

16. The controller of claim 15, wherein the pump drive signal reflects a blade position of an impeller of the mechanical circulatory support system.

17. The controller of claim 15, wherein the pump drive signal comprises a signal received from a transistor gate of motor drive circuitry of the mechanical circulatory support system.

18. The controller of claim 15, wherein the one or more light pulses comprises a plurality of light pulses that collectively produce stroboscopic flashing by the one or more light sources.

19. The controller of claim 15, wherein the one or more light sources include a light emitting diode (LED) source.

20. The controller of claim 15, wherein the process further comprises:
selecting one or more characteristics of the one or more light pulses based, at least in part, on a speed of a motor associated with the mechanical circulatory support system,
wherein the one or more characteristics include one or more of a pulse width, a pulse delay, or a pulse amplitude.
